(19) United States
(12) Patent Application Publication
(10) Pub. No.: US 2011/0029039 A1
(43) Pub. Date: Feb. 3, 2011

(54) Intracranial Electrical Seizure Therapy (ICEST)

(76) Inventor: Oscar Morales, Boston, MA (US)

Correspondence Address: FISH & RICHARDSON P.C. (BO), P.O. BOX 1022, MINNEAPOLIS, MN, 55440-1022, US

(21) Appl. No.: 12/743,139

(22) PCT Filed: Nov. 17, 2008
(86) PCT No.: PCT/US08/83732

§ 371 Date: Oct. 22, 2010

Related U.S. Application Data
(60) Provisional application No. 60/988,524, filed on Nov. 16, 2007.

(51) Int. Cl. A61N 1/36

(52) U.S. Cl. 607/45

(57) ABSTRACT

A method and system for administering intracranial electroconvulsive therapy are described. The method includes implanting an electrode at a target site in the brain, the electrode being connected to a controller; configuring the controller to deliver an electrical stimulus sufficient to induce a seizure that starts at the target site and spreads throughout a localized or generalized volume of the brain; and delivering the electrical stimulus through the electrode.

### CROSS REFERENCE TO RELATED APPLICATION

This application claims priority, under 35 U.S.C. §119(e), from U.S. Provisional Patent Application Ser. No. 60/988,524 filed on Nov. 16, 2007, which is incorporated herein by reference in its entirety.

### TECHNICAL FIELD

This disclosure relates to treating neuropsychiatric disorders by delivering electrical stimulation to the brain and inducing a seizure.

### BACKGROUND

Electroconvulsive therapy (ECT), also known as electroshock therapy, is a form of psychiatric treatment in which seizures are induced with electricity for therapeutic effect. ECT is generally used to treat severe depression and other neuropsychiatric disorders.

In carrying out ECT, electrodes are placed on a patient's head (scalp), e.g., one on either side of the patient's head, and electrical stimuli are delivered to the patient's brain through the electrodes. The application of electrical stimuli to many different parts of the brain induces transcranial seizures, which can span large portions and different areas of the brain. Some known side-effects of ECT include confusion and memory loss.

### SUMMARY

Intracranial electrical seizure therapy (ICEST) is an alternative method of treatment for those individuals suffering from chronic and severe depression and possibly for other neuropsychiatric disorders.

In general, in one aspect, the invention features a method for administering intracranial electroconvulsive therapy. The method includes implanting an electrode at a target site in the brain, the electrode being connected to a controller; configuring the controller to deliver an electrical stimulus sufficient to induce a seizure that starts at the target site and spreads throughout a localized or generalized volume of the brain; and delivering the electrical stimulus through the electrode.

In general, in another aspect, the invention features a method for treating a neuropsychiatric disorder. The method includes implanting an electrode at a target site in the brain, the electrode being connected to a controller; configuring the controller to deliver an electrical stimulus sufficient to induce a seizure that starts at the target site and spreads throughout a localized or generalized volume of the brain; and delivering the electrical stimulus through the electrode.

In general, in a further aspect, the invention features a system for administering intracranial electroconvulsive therapy. The system includes an electrode configured to be implanted at a target site in the brain; and a controller electrically coupled to the electrode, the controller configured to deliver an electrical stimulus through the electrode to induce a seizure that starts at the target site and spreads throughout a localized or generalize volume of the brain.

Embodiments may include one or more of the following. One or more electrical parameters may be delivered to the controller from a programmer over a wireless network, where the electrical parameters determine a dosage and the physical properties of the electrical stimulus. One or more leads connecting the electrode to the controller may be implanted subcutaneously in a subject undergoing treatment. The disorder may, for example, be one of depression, bipolar affective disorder, schizoaffective disorder, obsessive compulsive disorder, eating disorder with comorbid affective disorder, Parkinson's disease, Alzheimer's disease, Huntington's disease, Amyotrophic Lateral Sclerosis, stroke, brain neurodegenerative disorders, cortical and subcortical dementias, disorders characterized by brain tissue, neuron cell loss or degeneration. A condition of a subject undergoing treatment for the neuropsychiatric disorder may be monitored and one or more properties of the electrical stimulus may be adjusted based on feedback information acquired from the subject. The electrode and the controller may be integrated into a single device that is implanted subcutaneously in a subject.

The details of one or more embodiments are set forth in the accompanying drawings and the description below. Other features, objects, and advantages will be apparent from the description and drawings, and from the claims.

### DESCRIPTION OF DRAWINGS

FIG. 1 is a block diagram of an intracranial electrical seizure therapy (ICEST) system.

FIG. 2 is a block diagram of a controller for use with ICEST system of FIG. 1.

FIG. 3 is flow chart of a treatment process using the ICEST system of FIG. 1.

### DETAILED DESCRIPTION

Intracranial electrical seizure therapy (ICEST) provides treatment for chronic depression and other neuropsychiatric disorders by inducing a seizure with an electrode implanted in the brain. The electrode delivers electrical stimulation to a target site in the brain to induce a seizure that starts at the target site and spreads throughout a localized or generalized volume of the brain. The focal application of the electrical stimulation reduces the exposure of non-targeted brain areas to the applied stimulation, which in turn reduces side effects resulting from the electrical stimulation of these non-targeted areas.

The seizures induced by ICEST are believed to cause a variety of physiological effects that would provide neurotherapeutic intervention for neuropsychiatric conditions characterized by neuron cell loss or degeneration. These effects include the regulation of neuron cell growth factors expression, axonal sprouting, endothelial cell proliferation and neurogenesis. Medical conditions that can potentially respond to or be treated by ICEST include among others: depressive disorder, bipolar affective disorder, schizoaffective disorder, obsessive compulsive disorder, eating disorder with comorbid affective disorder, Parkinson's disease, Alzheimer's disease, Huntington's disease, Amyotrophic Lateral Sclerosis, stroke, brain neurodegenerative disorders, cortical and subcortical dementias, disorders characterized by brain tissue, neuron cell loss or degeneration.

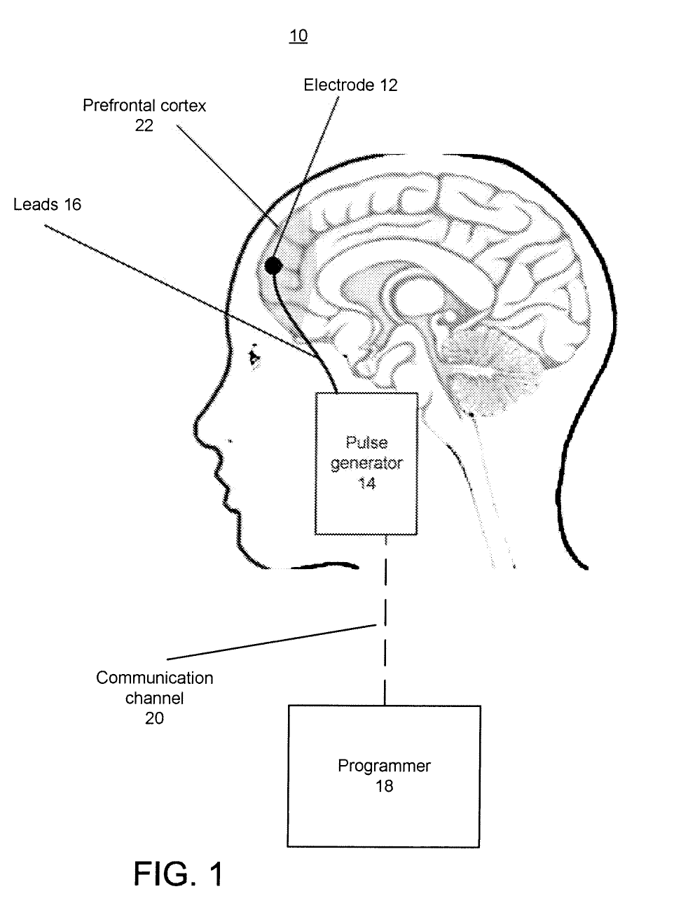

Referring to FIG. 1, an ICEST system 10 includes an electrode 12, which is surgically implanted at a target site in the brain, one or more leads (“leads 16”) connected to the electrode 12, a pulse generator 14 connected to the leads, and a programming device (“programmer 18”) in communication with the pulse generator 14 via a communication channel 20. The electrode 12 is a conductive material (e.g., a metal or metal alloy) that delivers electrical signals directly to the implantation site. In some embodiments, more than one electrode 12 may be implanted in the brain.

The pulse generator 14 generates the electrical signals according to stimulation parameters supplied by the programmer 18, and the leads 16 carry the electrical signals from the pulse generator 14 to the electrode 12. Like the electrode 18, the leads 16 are composed of a conductive material (e.g., a metal or metal alloy). For example, the leads 16 can be wires. In some embodiments, the leads 16 and electrode 18 are integrated, in which case the electrode 18 comprises those portions of the leads 16 that are implanted at the target site.

FIG. 1 shows one possible target site residing in the prefrontal cortex 22 of the brain. The target site may be located in any area of the prefrontal cortex 22, e.g., the subdural area; as well as in other areas of the cortex. The target site may also be located in other areas of the brain besides the cortex.

The programmer 18 sends selected electrical parameters, which represent the electrical stimulation required to initiate or induce a seizure at the target site, to the pulse generator 14 over the communication channel 20. In some embodiments, the programmer 18 calculates the parameters based on input supplied by a skilled practitioner. In other embodiments, the programmer 18 receives the parameters as direct input from a skilled practitioner or another entity (e.g,. a computer). The programmer 18 may be a standard ECT machine or another computer-based system. The communication channel 20 may be a physical connection, e.g., a wire that plugs into one or both of the pulse generator 14 and the programmer 18, or a wireless connection, e.g., an RF or infrared link between the pulse generator 14 and the programmer 18. The communication channel 20 may also be a network, e.g., a LAN, a WAN, and/or the Internet.

In some embodiments, the pulse generator 14 is an external device that resides outside of the patient's body. In other embodiments, the pulse generator 14 is an internal device that is implanted inside the patient, e.g., subcutaneously in the chest, with all of the leads 16 also implanted subcutaneously in the patient's body. In further embodiments, the pulse generator 14, leads 16, and electrode 12 are integrated as a single device, e.g., a micro chip-electrode-stimulator, that can be implanted in the brain and programmed by wireless telemetry to in induce stimulation that implements ICEST. In embodiments in which the pulse generator 14 is internal to the patient, the communication channel 20 is generally a wireless connection and the stimulation parameters may be delivered telemetrically from the programmer 18 to the pulse generator 14.

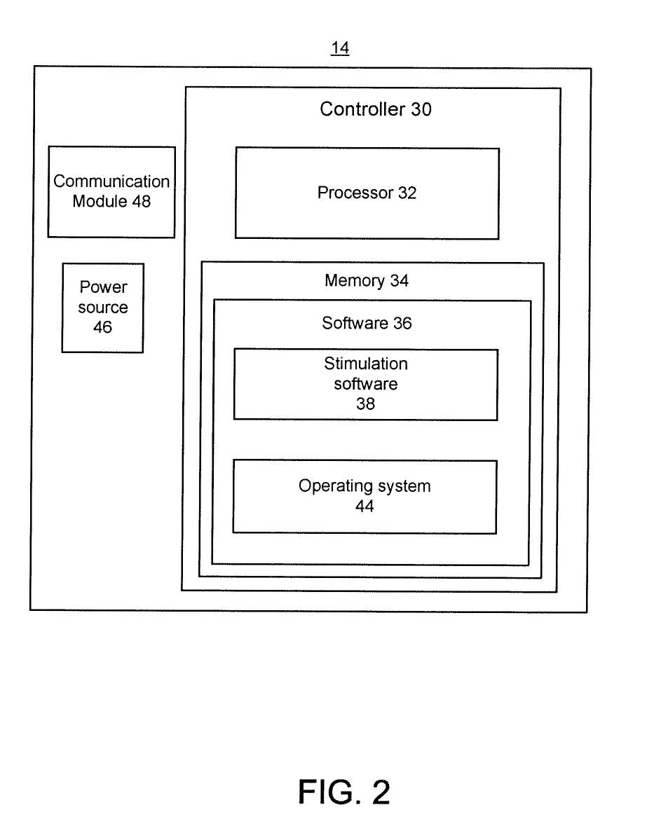

Referring to FIG. 2, a block diagram shows the pulse generator 14 of the ICEST system 10 in further detail. The pulse generator 14 includes a controller 30, a communication module 48 for affecting communication with the programmer 18 over the communication channel 20, and a power source 46 (e.g., a battery) for providing power to the controller 30. The controller 30 includes one or more processor(s) 32 (referred to simply as “processor 32”) and memory 34 for storing software 36. The processor 32 executes software 36, which includes stimulation software 38 and an operating system 44 (e.g., such as, UNIX or Windows XP®).

The stimulation software 38 includes programmable stimulation parameters that control the characteristics and dosage of electricity delivered to the target site. Examples of stimulation parameters include pulse width, frequency, voltage, current, and a duration of time over which the electricity is applied. The stimulation parameters are initially programmed by the programmer 18 via commands sent over the communication channel 20 to the pulse generator 14. The parameters may later be adjusted using the same or a similar programming mechanism. For example, the parameters may be adjusted based on feedback received from the patient and/or based on the observations of a skilled practitioner.

The communication module 48 includes the necessary hardware and software for implementing a communication protocol (e.g., a TCP/IP protocol) to enable the controller 30 or any components thereof, to communicate with the programmer 18 over a wired or wireless communication channel 20. The communication module 28, for example, could include an Ethernet modem and/or a wireless modem.

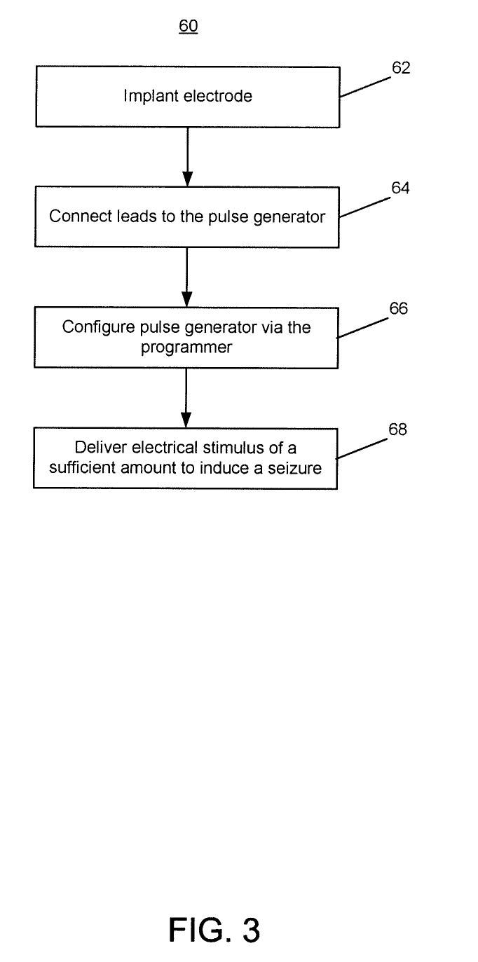

Referring to FIG. 3, a process 60 for treating a patient using ICEST is performed using the system 10 shown in FIG. 1. The electrode 12 is surgically implanted in the brain at a target site (step 62). For example, in some embodiments, the electrode 12 is implanted in the prefrontal cortex of the brain. The leads 16, which are attached to the electrode 12, are also surgically implanted in the patient and connected to the pulse generator 14 (step 64). The pulse generator 14 and leads 16 may also be connected prior to implantation of the electrode 12. In some embodiments, the pulse generator 14 is also implanted into the patient. In these embodiments, the pulse generator 14 uses a wireless connection to communicate with the programmer 18. In other embodiments, the pulse generator 14 resides outside of the patient. As an external device, the pulse generator 14 may be plugged directly into the programmer 18, e.g., via a wire, or communicate over a wireless connection.

The pulse generator 14 is configured by the programmer 18 (step 66). During configuration, the pulse generator 14 receives, from the programmer 18, preselected stimulation parameters that describe a dosage of electrical stimulus to be applied to the patient at the electrode 12. These parameters describe various electrical characteristics of the electrical stimulus and a period of duration. The stimulation parameters, which may vary among patients, are selected to be sufficient to induce a seizure in the patient. Examples of a range of stimulation parameters that may be suitable for some patients are the following: pulse width 0.1 to 3 milliseconds, frequency of a pulse 10 Hz to 200 Hz (Hertz), duration 100 milliseconds to 6 seconds, current of approximately 750 to 850 mA.

Under control of an operator, the pulse generator 14 then delivers (step 68) the electrical stimulus to the target site of the patient's brain. The leads 16 then carry the stimulus from the pulse generator 14 to the electrode 12, where it makes contact with the patient's brain. Once delivered, the stimulus induces a seizure that starts at the target site and spreads throughout a localized or generalized volume. In some embodiments, the localized volume consists of the prefrontal cortex, other cortical, subcortical areas, and deep structures. The brain seizure is measured by electroencephalography. The seizure induction is performed following an anesthetic procedure similar to that used in conventional ECT, which involves intravenously administering to the patient a general short acting anesthetic and a short acting muscular relaxant.

After the seizure subsides, the patient gradually recovers from the anesthesia and treatment until he recovers breathing function and regains his previous state of awareness and alertness. After the seizure subsides, a medical practitioner evaluates the patient's condition. For example, the practitioner may run a series of medical tests on the patient and/or interview the patient.

The ICEST process 60 is usually performed several times weekly and the total number of treatment sessions may depend on the primary condition subject of treatment, severity of the patient's symptoms and rapidity of response. The ICEST process 60 may be repeated several times a week to achieve a desired effect, e.g., until the patients symptoms have improved or have completely subsided. The ICEST process 60 may be performed again later if the patient's symptoms worsen or reappear. After remission of symptoms a maintenance therapy may be required.

The techniques described herein can be implemented in digital electronic circuitry, or in computer hardware, firmware, software, or in combinations of them. The techniques can be implemented as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Method steps of the techniques described herein can be performed by one or more programmable processors executing a computer program to perform functions of the invention by operating on input data and generating output. Method steps can also be performed by, and apparatus of the invention can be implemented as, special purpose logic circuitry, e.g., an FPGA (field programmable gate array) or an ASIC (application-specific integrated circuit). Modules can refer to portions of the computer program and/or the processor/special circuitry that implements that functionality.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

To provide for interaction with a user, the techniques described herein can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element, for example, by clicking a button on such a pointing device). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The techniques described herein can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an implementation of the invention, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks.

The following are examples for illustration only and not to limit the alternatives in any way. The techniques described herein can be performed in a different order and still achieve desirable results. Other embodiments are within the scope of the following claims.

What is claimed is:

1. A method for administering intracranial electroconvulsive therapy, the method comprising:
implanting an electrode at a target site in the brain, the electrode being connected to a controller;
configuring the controller to deliver an electrical stimulus sufficient to induce a seizure that starts at the target site and spreads throughout a localized or generalized volume of the brain; and
delivering the electrical stimulus through the electrode.

2. The method of claim 1, wherein the electrode is implanted in the brain cortex, subcortical area or deep structure.

3. The method of claim 1, further comprising delivering one or more electrical parameters to the controller from a programmer over a wireless network, the electrical parameters determining a dosage and physical properties of the electrical stimulus.

4. The method of claim 3, further comprising selecting the electrical parameters to include a pulse width between 0.1 to 3.0 milliseconds, a pulse frequency between 100 Hz to 200 Hz (Hertz), a pulse duration between 100 milliseconds to 6 seconds, and a current of approximately 750 to 850 mA.

5. The method of claim 1, further comprising implanting one or more leads subcutaneously in a subject undergoing treatment, the leads connecting the electrode to the controller.

6. A method of treating a neuropsychiatric disorder, the method comprising:
implanting an electrode at a target site in the brain, the electrode being connected to a controller;
configuring the controller to deliver an electrical stimulus sufficient to induce a seizure that starts at the target site and spreads throughout a localized or generalized volume of the brain; and
delivering the electrical stimulus through the electrode.

7. The method of claim 6, wherein the disorder is depression.

8. The method of claim 6, wherein the disorder is Alzheimer's disease, Parkinson's disease, Huntington's disease, Amyotrophic Lateral Sclerosis, stroke, brain neurodegenerative disorders, cortical and subcortical dementias, disorders characterized by brain tissue, or other conditions characterized by neuron cell loss or degeneration.

9. The method of claim 6, wherein the disorder is one of bipolar affective disorder, schizoaffective disorder, obsessive compulsive disorder, and eating disorder.

10. The method of claim 6, further comprising delivering one or more electrical parameters to the controller from a programmer over a wireless network, the electrical parameters determining a dosage and physical properties of the electrical stimulus.

11. The method of claim 6, further comprising:
monitoring a condition of a subject undergoing treatment for the neuropsychiatric disorder; and
adjusting one or more properties of the electrical stimulus based on feedback information acquired from the subject.

12. The method of claim 11, wherein the feedback information is based on one or more observations of the subject and a skilled practitioner monitoring the subject.

13. The method of claim 11, wherein monitoring comprises measuring the seizure using electroencephalography.

14. A system for administering intracranial electroconvulsive therapy, the system comprising:
an electrode configured to be implanted at a target site in the brain; and
a controller electrically coupled to the electrode, the controller configured to deliver an electrical stimulus through the electrode to induce a seizure that starts at the target site and spreads throughout a localized or generalized volume of the brain.

15. The system of claim 14, further comprising one or more leads connecting the electrode to the controller, the leads configured to be implanted subcutaneously in a subject.

16. The system of claim 14, further comprising a programmer wirelessly coupled to the controller, the programmer configured to deliver one or more selected electrical parameters to the controller over a wireless network, the electrical parameters determining a dosage and physical properties of the electrical stimulus.

17. The system of claim 16, wherein the electrical parameters include a pulse width between 0.1 to 3.0 milliseconds, a pulse frequency between 100 Hz to 200 Hz (Hertz), a pulse duration between 100 milliseconds to 6 seconds, and a current of approximately 750 to 850 mA.

18. The system of claim 14, wherein the electrode and the controller are integrated into a single device configured to be implanted subcutaneously in a subject.
